(19) United States
(12) Patent Application Publication
(10) Pub. No.: US 2005/0256196 A1
(43) Pub. Date: Nov. 17, 2005

(54) Decreasing brain neuronal glutamate levels using alpha-keto branched chain amino acids

(75) Inventor: William S. Brusilow, Grosse Pointe, MI (US)

Correspondence Address: ROTHWELL, FIGG, ERNST & MANBECK, P.C., 1425 K STREET, N.W., SUITE 800, WASHINGTON, DC, 20005, US

(73) Assignee: Odessa Pharma, Chevy Chase, MD (US)

(21) Appl. No.: 11/130,214

(22) Filed: May 17, 2005

Related U.S. Application Data
(60) Provisional application No. 60/571,523, filed on May 17, 2004.

(51) Int. Cl. A61K 31/198; A61K 31/19

(52) U.S. Cl. 514/557; 514/562

(57) ABSTRACT

The present invention relates to the treatment or prevention of glutamatergic toxicity by the administration of effective amounts of branched chain α-keto acids alone or in combination with other antiglutamate agents such as L-methionine S-sulfoximine, L-ethionine S-sulfoximine, and glufosinate. In particular, the present invention relates to the treatment or prevention of diseases or conditions which are characterized by increased levels of brain neuronal glutamate.

### FIELD OF THE INVENTION

The present invention relates, generally, to the treatment or prevention of glutamatergic toxicity by the administration of effective amounts of branched chain α-keto acids, L-methionine S-sulfoximine (MSO), L-ethionine S-sulfoximine, and/or glufosinate. In particular, the present invention relates to the treatment of diseases or conditions in which the toxic excitogenic effect of accumulated glutamate is implicated. Such diseases and conditions include but are not limited to amyotrophic lateral sclerosis, Alzheimer's disease, epilepsy, stroke, multiple sclerosis, schizophrenia and hypoxia-ischemia.

### DESCRIPTION OF RELATED ART

Glutamate is a five-carbon skeleton dicarboxylic amino acid which is the principal excitatory neurotransmitter in the mammalian central nervous system. Glutamate activates metabotropic glutamate receptors (mGluRs) which are coupled to a variety of signal transduction pathways via guanine-nucleotide-binding proteins (G proteins). Glutamate also binds to a variety of excitatory amino acid receptors, which are ligand-gated ion channels. It is the activation of these receptors that leads to depolarisation and neuronal excitation. In normal synaptic functioning, activation of excitatory amino acid receptors is transitory. However, if, for any reason, receptor activation becomes excessive or prolonged, such as when the glutamate concentration in the extracellular fluid becomes elevated, the target neurons become damaged and eventually die. This process of neuronal death is called excitotoxicity. Excitotoxicity resulting from extracellular accumulation of endogenous glutamate is a major contributor to neuronal death. Glutamate excitotoxicity is characterized by increasing damage of cell components, including mitochondria, leading to cell death.

The blood-brain barrier excludes many amino acids from neurons and thus specific physiologic methods have evolved to provide amino acids to neurons. Released glutamate is taken up by brain glia cells, converted to glutamine by the enzyme glutamine synthetase, transported to the presynaptic neuron and then converted back to glutamate via intraneuronal deamidation of glutamine. In this process, glutamine is synthesized from ammonia and glutamate catalyzed by glutamine synthetase in astrocytes, the only brain cells rich in glutamine synthetase activity. The glutamine so produced is transported to neurons where it serves two roles; 1) it is used for protein synthesis (e.g. Huntingtin) and 2) it is deamidated by neuronal glutaminase to provide glutamate which serves many functions one of which is a neurotransmitter. To avoid its accumulation and glutamatergic toxicity, glutamate is returned to the astrocytes by its transporters and resynthesized to glutamine to repeat the cycle. Neuronal glutamate is transported by neuronal transporters to the synaptic cleft from where it transported back to the astrocytes by astrocyte transporters (Reisberg B, Doody R, Stoffler A, et al. Memantine in moderate-to-severe Alzheimers disease. N Engl J Med 2003:348;1333-1341) where it is synthesized back to glutamine in a reaction catalyzed by glutamine synthetase.

Glutamate is also the product of many transaminases in all tissues, including the brain. The amino group from amino acids is transferred to α-ketoglutarate to form glutamate and the cognate α-keto acid. For example, leucine aminotransferase (or leucine transaminase) reacts leucine with α-ketoglutarate to form glutamate and α-keto isocaproate. These reactions are freely reversible. The direction is driven by mass action—the relative concentrations of reactants and products.

Disruptions in the glutamate-glutamine cycle have been implicated in a number of neuro-degenerative diseases such as amyotrophic lateral sclerosis (Heath P R, Shaw P J. Update on the glutamatergic neurotransmitter system and the role of excitotoxicity in amyotrophic lateral sclerosis. Muscle and Nerve 2002:36;438-458), Alzheimer's disease, epilepsy, and stroke (Maragakis N J, Rothstein J D. Glutamate transporters in neurologic disease. Arch Neurol 2001:58; 365-370), schizophrenia (Tsai G, Coyle J T. Glutamatergic mechanisms in schizophrenia. Ann Rev Pharmacol Toxico 2002:42;165-179), and multiple sclerosis (Sarchielli, P., Greco, L., Floridi, A., Floridi, A., Gallai, V. 2003. Excitatory amino acids and multiple sclerosis. Arch. Neurol.:60, 1082-1088). In addition, premature infants are particularly vulnerable to excitotoxicity which can result in long term neurologic abnormalities such as cerebral palsy and epilepsy (Koh S, Jensen F E. Topiramate blocks seizures in a rodent model of perinatal hypoxic encephalopathy. Ann Neurol 2001;50:366

-72).

Attempts at anti-glutamatergic therapy have been proposed (Kim A H, Kerchner G A, Choi D W. Blocking excitotoxicity. In; Marcoux F W, Choi D W, eds. CNS Neuroprotection. Berlin:Springer 2002:3-36; Reisberg B, Doody R, Stoffler A, et al. Memantine in moderate-to-severe Alzheimer's disease. N Engl J Med 2003:348;1333-1341) but these attempts do not manipulate glutamine metabolism or the concentration of brain glutamate. Other drugs which antagonize the N-methyl-D-aspartate (NMDA) receptor have been proposed but have generally not been successful (Fogarty, M., Targeting Excitotoxicity, Preclinica, vol. 2, No. 1, 2004).

### SUMMARY OF THE INVENTION

The purpose of this invention is to reduce the availability of free glutamate and glutamine in astrocytes, the source of glutamine and ultimately, to reduce the availability of glutamate for neurons. Reducing the availability of glutamate will aid in the treatment or prevention of glutamatergic toxicity. The object of the present invention is achieved by the administration of effective amounts of branched chain α-keto acids, L-methionine S-sulfoximine, L-ethionine S-sulfoximine, and/or glufosinate. In particular, the present invention relates to the treatment or prevention of diseases and conditions in which the toxic excitogenic effect of accumulated glutamate is implicated. Such diseases include but are not limited to amyotrophic lateral sclerosis, Alzheimer's disease, epilepsy, stroke, multiple sclerosis and schizophrenia. The present invention is also useful for reducing brain injury in infants who have experienced a traumatic birth event that compromised the brain's blood and oxygen supply (e.g. hypoxia-ischemia).

Further features and advantages of the present invention, are described in detail below.

### DETAILED DESCRIPTION OF THE INVENTION

In addition to the glutamine-glutamate cycle between neurons and astrocytes, there has been proposed a leucine-glutamate cycle (Daikhin, Y., and Yudkoff, 2000. M. Compartmentation of brain glutamate metabolism in neurons and glia. J. Nutrition 130, 1026S-1031S; Yudkoff et al. 1994, Interrelationships of leucine and glutamate in cultured astrocytes. J. Neurochemistry. 62; 1192-1202), in which leucine, and other branched-chain amino acids, cross the blood brain barrier (Conn, A. R., and Steele, R. D. 1982. Transport of α-keto analogues of amino acids across blood-brain barrier in rats. Am. J. Physiol. Endocrinol. Metab. 243:E272-E277; Steele, R. D. 1986. Blood-brain barrier transport of the α-keto analogs of amino acids, Federation Proceedings 45: 2060-2064) and are transaminated by α-ketoglutarate to glutamate and the cognate α-keto acid in astrocytes, as a mechanism for forming glutamate. The glutamate is then amidated by glutamine synthetase to form glutamine, which leaves the astrocyte and is transported into the neuron where it is hydrolyzed to glutamate and ammonia. Neuronal glutamate can be oxidized by glutamate dehydrogenase, or be transaminated by α-keto acids to form α-ketoglutarate and the cognate amino acid. The α-ketoglutarate can be oxidized by mitochondrial metabolism, and the amino acid released from the neuron, where it can either cross the blood-brain barrier or be taken up again by the astrocyte.

The α-keto derivatives of the three branched-chain amino acids leucine, valine, and isoleucine can substitute for these three essential amino acids in the diet. They cross the blood brain barrier, where they are transaminated by glutamate to form the cognate amino acid and α-keto-glutarate, thus reducing brain glutamate levels. The resultant amino acids can be used for protein synthesis, or can cross the blood brain barrier and be transported throughout the body, where they will subsequently be used for protein synthesis or metabolized to other compounds. Since brain glutamine and glutamate do not cross the blood-brain barrier in appreciable amounts compared to their concentrations inside the brain, the net result is a reduction of brain glutamate, thereby reducing the amount of glutamine synthesized by astrocytes.

Administration of the salts of the a-keto acids of leucine, isoleucine, and valine—α-keto isocaproate, α-keto-methyl butyrate, and α-keto isovalerate (doses of about 100-500 mg/kg body weight, preferably 280-380 mg/kg body weight per day), respectively, will drive the transaminase reactions in the direction of α-ketoglutarate synthesis, thus reducing neuronal glutamate levels. The latter effect, is beneficial in those diseases in which the toxic excitogenic effect of accumulated glutamate is implicated (including but not limited to amyotrophic lateral sclerosis, Alzheimer's disease, epilepsy, stroke, multiple sclerosis, schizophrenia and hypoxia-ischemia). Branched-chain α-keto acids have been tested for efficacy in the treatment of hyperammonemia and of hepatic encephalopathy (Walser, M. 1984. Therapeutic aspects of branched-chain amino and keto acids. Clinical Sci. Mol. Med 66: 1-15), with little or no success, but have not been used in the treatment of excitoxicity as in the present invention.

The α-keto acids of the present invention can be administered in combination with L-methionine S-sulfoximine (MSO) which inhibits glutamine synthetase, an astrocyte specific enzyme (Martinez-Hernandez A, Bell K P, Norenberg M D. Glutamine synthetase: Glial localization in brain. Science 1977:195;1356-1358), which catalyzes the biosynthesis of glutamine from ammonia and glutamate (Lamar C., The duration of the inhibition of glutamine synthetase by methionine sulfoximine, Biochem Pharm 1968:17;636-640) and which is known to reduce brain astrocyte levels of glutamine by 66% in normal animals and reduce nerve terminal glutamate levels by 52% (Laake J H, Slyngstad T A, Haug F S, Otterson O P. Glutamine from glial cells is essential for the maintenance of the nerve terminal pool of glutamate: Immunogold evidence from hippocampal slice cultures. J Neurochem 1995:65;871-881). MSO, when administered to normal animals, reduced brain levels of glutamine from 5.6 to 1.8 (mmol/kg brain) (AM J. physiol 1991: 261; H825-829). A patient's brain glutamate levels should be monitored before, during and after treatment by any suitable method including determining glutamate levels in cerebrospinal fluid. The dosage can then be adjusted according to each patient's needs.

MSO can be administered at a dose of 2.0-40.0 mg/kg per 6-10 days (orally or intravenously) or it can be administered intrathecally at a dose of 1.0-5.0 mg per 6-10 days. This dosage has two effects. It will result in a reduction in the astrocyte pool of glutamine and, consequently, the pool of neuronal glutamate (Laake J H, Slyngstad T A, Haug F S, Otterson O P. Glutamine from glial cells is essential for the maintenance of the nerve terminal pool of glutamate: Immunogold evidence from hippocampal slice cultures. J Neurochem 1995:65;871-881) thereby reducing its toxic excitogenic effect. The latter effect is beneficial in those diseases and conditions in which the toxic excitogenic effect of accumulated glutamate is implicated (amyotrophic lateral sclerosis, Alzheimer's disease, epilepsy, stroke, schizophrenia and hypoxia-ischemia).

In addition to its glutamine synthetase inhibitory effect, MSO can cause periodic convulsions depending on the dosage. Two strategies can be employed to prevent this convulsant effect. One strategy is to use lower doses of MSO, in some species notably primates and presumably including man, a lower dose preserves the glutamine synthetase inhibitory activity while avoiding convulsant activity. If a higher dosage is required, L-methionine can be simultaneously administered along with the MSO. Sellinger et al. (Methionine sulfoximine seizures. VIII. The dissociation of the convulsant and glutamine synthetase inhibitory effects. Journal Pharmacology and Experimental Therapeutics. 164:212-222;1968) has shown that the simultaneous administration of L-methionine (MW 148) along with known convulsant doses of MSO (MW 179) to rats, preserves the glutamine synthetase inhibitory activity but prevents the convulsant activity. The L-methionine is administered at a dosage (on a molar basis) of 5 mmol of L-methionine per mmol MSO or 4 mg L-methionine per mg MSO.

Glufosinate, ammonium-D-L-homoalanine-4-yl(methyl)-phosphinate, (Hack R, Ebert E, Leist H. Glufosinate ammonium-some aspects of its mode of action in mammals. Fd Chem Toxic 1994:32;461-470) is also a catalytic inhibitor of glutamine synthetase which can be administered in combination with a-keto acids. Glufosinate must be administered intrathecally at a dose of 1.0-5.0 mg per 6-10 days because it does not cross the blood-brain barrier. However, when administered in this manner, it operates by the same mechanism (Gill H S, Eisenberg D. The crystal structure of phosphinothricin in the active site of glutamine synthetase illuminates the mechanism of enzymatic inhibition. Biochemistry 2001:440;1903-1912) as does MSO describe above. Prior to the present invention, neither animals, humans nor in vitro preparations with these diseases have been treated with glufosinate.

One or more compounds according to the present invention can be formulated into compositions along with other compounds known to be useful for the treatment of glutamatergic toxicity. Such compounds include but are not limited to neuroprotective compounds and other antiglutamate agents. Examples of such compounds include but are not limited to riluzole, remacemide, amantadine, memantine, gabapentin, lithium, topiramate and cystamine. The compounds according to the present invention can also be included in kits which comprise L-methionine S-sulfoximine, L-ethionine S-sulfoximine, glufosinate and/or branched chain α-keto acids in separate containers along with other compounds known to be useful for the treatment of glutamatergic toxicity in separate containers. The compounds can be mixed to produce custom formulated compositions or the compounds can be administered separately.

The compounds can be administered prior to the appearance of symptoms in order to delay or prevent the occurrence of symptoms.

The formulations of the present invention include those suitable for oral, rectal, nasal, inhalation (e.g., to the lungs), buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder. Formulations suitable for oral administration may be controlled release or osmotic dosage forms.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for nasal, parenteral, or inhalation administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound, in a unit dosage form in a sealed container. The compound or salt is provided in the form of a lyophilizate which is capable of being reconstituted with a suitable pharmaceutically acceptable carrier to form a liquid composition suitable for injection thereof into a subject. When the compound or salt is substantially water-insoluble, a sufficient amount of emulsifying agent that is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis/tris buffer (pH 6) or ethanol/water and contain from 0.1 to 0.2M active ingredient.

Further, the present invention provides liposomal formulations of the active compounds disclosed herein. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

The liposomal formulations containing the compounds disclosed herein, may be lyophilized to produce a lyophilizate which may be reconstituted with a pharmaceutically acceptable carrier, such as water, to regenerate a liposomal suspension.

Other pharmaceutical compositions may be prepared from the compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compounds, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

The therapeutically effective dosage or treatment effective amount of any one active agent will vary somewhat from compound to compound as discussed above, and patient to patient (depending upon the age and condition of the subject), and will depend upon factors such as the age and condition of the patient and the route of delivery. Such dosages can be determined in accordance with routine pharmacological procedures known to those skilled in the art. The compounds can be administered prior to the appearance of symptoms in order to delay or prevent the occurrence of symptoms.

Administration may be carried out on a chronic or acute basis. When the administering step is an acute administering step, the active agent may (for example) be given as a single dosage as above, or daily in the above dosages for a period of 6-10 days. Where the administering step is a chronic administrating step, the daily dosage will be given at least 3, 4 or 5 times a week (e.g., seven days a week) for a period of at least two weeks, at least a month, at least two months, or even at least six months or more. When a chronic dosage regimen is completed the patient may be reevaluated and the administration continued or modified as necessary.

As used herein, the term “administering to the brain of a subject” refers to the use of routes of administration, as are known in the art, that provide the therapeutic compound to the central nervous system tissues, and in particular the brain, of a subject being treated.

The blood-brain barrier presents a barrier to the passive diffusion of substances from the bloodstream into various regions of the CNS. However, active transport of certain agents is known to occur in either direction across the blood-brain barrier. Substances that may have limited access to the brain from the bloodstream can be injected directly into the cerebrospinal fluid. Cerebral ischemia and inflammation are also known to modify the blood-brain barrier and result in increased access to substances in the bloodstream.

Administration of a therapeutic compound directly to the brain is known in the art. Intrathecal injection administers agents directly to the brain ventricles and the spinal fluid. Surgically-implantable infusion pumps are available to provide sustained administration of agents directly into the spinal fluid. Lumbar puncture with injection of a pharmaceutical compound into the cerebrospinal fluid (“spinal injection”) is known in the art, and is suited for administration of the present therapeutic compounds.

Pharmacologic-based procedures are also known in the art for circumventing the blood brain barrier, including the conversion of hydrophilic compounds into lipid-soluble drugs. The active agent may be encapsulated in a lipid vesicle or liposome.

The intra-arterial infusion of hypertonic substances to transiently open the blood-brain barrier and allow passage of hydrophilic drugs into the brain is also known in the art. U.S. Pat. No. 5,686,416 to Kozarich et al. discloses the co-administration of receptor mediated permeabilizer (RMP) peptides with therapeutic compounds to be delivered to the interstitial fluid compartment of the brain, to cause an increase in the permeability of the blood-brain barrier and effect increased delivery of the therapeutic compounds to the brain. Intravenous or intraperitoneal administration may also be used to administer the compounds of the present invention.

One method of transporting an active agent across the blood-brain barrier is to couple or conjugate the active agent to a second molecule (a “carrier”), which is a peptide or non-proteinaceous moiety selected for its ability to penetrate the blood-brain barrier and transport the active agent across the blood-brain barrier. Examples of suitable carriers include pyridinium, fatty acids, inositol, cholesterol, and glucose derivatives. The carrier may be a compound which enters the brain through a specific transport system in brain endothelial cells. Chimeric peptides adapted for delivering neuropharmaceutical agents into the brain by receptor-mediated transcytosis through the blood-brain barrier are disclosed in U.S. Pat. No. 4,902,505 to Pardridge et al. These chimeric peptides comprise a pharmaceutical agent conjugated with a transportable peptide capable of crossing the blood-brain barrier by transcytosis. Specific transportable peptides disclosed by Pardridge et al. include histone, insulin, transferrin, and others. Conjugates of a compound with a carrier molecule, to cross the blood-brain barrier, are also disclosed in U.S. Pat. No. 5,604,198 to Poduslo et al. Specific carrier molecules disclosed include hemoglobin, lysozyme, cytochrome c, ceruloplasmin, calmodulin, ubiquitin and substance P. See also U.S. Pat. No. 5,017,566 to Bodor.

Subjects to be treated by the methods of the present invention are typically human subjects, but may also be animal subjects (particularly mammalian subjects) such as dogs, cats, rats, mice, insects, etc., for veterinary purposes, or for drug design and screening purposes. The subjects may be afflicted with a disease or condition in which the toxic excitogenic effect of accumulated glutamate is implicated, or at risk of developing symptoms associated with the toxic excitogenic effect of accumulated glutamate, or a subject suspected of having high levels of brain neuronal glutamate.

The following examples are intended to illustrate but not limit the present invention.

### EXAMPLE 1

A cell based assay is used to show that α-keto branched chain amino acids reduce brain neuronal glutamate levels. Primary cultures of rat cerebral astrocytes are subjected to oxidative stress by incubation with tert-butyl hydroperoxide for 30 min, followed by a 30-90-min washout period. The effects of the administration of a -keto isocaproic acid-sodium salt (sodium ketoleucine) is determined by measuring the uptake of glutamate as well as the release of d-aspartate (a nonmetabolizable analog of glutamate) before and after the administration of α-keto isocaproic acid-sodium salt. The effect of α-keto isocaproic acid-sodium salt on excitotoxic cell death is determined by measuring lactic dehydrogenase (LDH) activity released into the culture medium.

### EXAMPLE 2

Male Sprague-Dawley rats are intraperitoneally administered either an α-keto isocaproic acid-sodium salt (sodium ketoleucine) solution or saline 24 hours before middle cerebral artery occlusion and decapitation 3 days later. The brains are removed and analyzed for infarcted gray matter. The infarct volumes can be determined as described in Swanson et al. (Methionine Sulfoximine Reduces Cortical Infarct Size in Rats After Middle Cerebral Artery Occlusion, Stroke, Vol 21, No. 2, February 1990).

### EXAMPLE 3

In order to show that the inhibition of glutamine by α-keto branched chain amino acids is decreasing brain neuronal glutamate levels, L-glutamine (L-GLN) is added to an α-keto branched chain amino acid treated culture of differentiated inducible PC12 cells (Proc Natl Acad Sci U S A. May 13, 2003; 100 (10): 5950-5955). The addition of L-glutamine reverses the decreased glutamine and glutamate levels due to the inhibitory effect of α-keto branched chain amino acids on the cells and results in increased glutamate levels. In contrast, the addition of L-GLN has no effect on the growth of cells not treated with a-keto branched chain amino acids or cultures treated with subinhibitory concentrations of a-keto branched chain amino acids.

1. A method for decreasing brain neuronal glutamate levels in a patient in need of such treatment, comprising administering branched chain a-keto acids derived from leucine, isoleucine or valine, to a patient in need of such treatment.

2. The method according to claim 1, wherein said patient is suffering from a disease or condition selected from the group consisting of amyotrophic lateral sclerosis, Alzheimer's disease, epilepsy, stroke, multiple sclerosis, schizophrenia and hypoxia-ischemia.

3. The method according to claim 1, wherein said compound is administered orally, intravenously, or intrathecally.

4. The method according to claim 1, wherein said branched chain α-keto acids derived from leucine, isoleucine or valine are administered orally at a dosage between 100-500 mg/kg body weight per 6-10 days.

5. The method according to claim 4, wherein said branched chain α-keto acids derived from leucine, isoleucine or valine are administered orally at a dose between 280-380 mg/kg body weight per 6-10 days.

6. The method according to claim 1, further comprising a compound selected from the group consisting of L-methionine S-sulfoximine, L-ethionine S-sulfoximine, and glufosinate.

7. The method according to claim 1, wherein said α-keto acid is selected from the group consisting of α-keto-isocaproate, α-keto-β-methylbutyrate and α-keto-valerate and salts thereof.

8. The method according to claim 1, further comprising administering a second compound which is selected from the group consisting of neuroprotective compounds and antiglutamate agents.

9. The method according to claim 8, wherein said second compound is selected from the group consisting of riluzole, remacemide, amantadine, memantine, gabapentin, lithium, topiramate and cystamine.

10. A composition comprising a) branched chain α-keto acids derived from leucine, isoleucine or valine, effective to reduce brain neuronal glutamate levels, b) a second antiglutamate agent, and c) a pharmaceutically acceptable carrier.

11. The composition according to claim 10, wherein said second compound is selected from the group consisting of riluzole, remacemide, amantadine, memantine, gabapentin, lithium, topiramate and cystamine.

12. The composition according to claim 10, further comprising a compound selected from the group consisting of L-methionine S-sulfoximine, L-ethionine S-sulfoximine, and glufosinate.

13. A kit comprising a) branched chain α-keto acids derived from leucine, isoleucine or valine, and b) one or more compounds selected from the group consisting of L-methionine S-sulfoximine, L-ethionine S-sulfoximine, and glufosinate; in separate containers.

14. The kit according to claim 13, further comprising another compound useful for treating glutamatergic toxicity.

15. The kit according to claim 14, wherein said compound useful for treating glutamatergic toxicity is selected from the group consisting of riluzole, remacemide, amantadine, memantine, gabapentin, lithium, topiramate and cystamine.

16. The kit according to claim 13, further comprising L-methionine.
